(19) United States
(12) Patent Application Publication
(10) Pub. No.: US 2025/0346942 A1
(43) Pub. Date: Nov. 13, 2025

(54) METHOD AND SYSTEM FOR ISOLATING AND PROFILING OF ONCOSOMES FOR EARLY DETECTION AND MONITORING OF ALL HUMAN CANCERS FROM PERIPHERAL BLOOD

(71) Applicant: Ramesh Vallabhaneni, Orlando, FL (US)

(72) Inventor: Ramesh Vallabhaneni, Orlando, FL (US)

(21) Appl. No.: 18/867,559

(22) PCT Filed: May 31, 2023
(86) PCT No.: PCT/US2023/067692

§ 371 Date: Nov. 20, 2024

Related U.S. Application Data
(60) Provisional application No. 63/348,686, filed on Jun. 3, 2022.

(51) Int. Cl. C12Q 1/6806; C12Q 1/6841; C12Q 1/6886

(52) U.S. Cl. CPC C12Q 1/6806; C12Q 1/6841; C12Q 1/6886

(57) ABSTRACT

A rapid non-invasive method for isolating Oncosomes, the extra cellular vesicles produced by the cancer cells, from human plasma is disclosed. Additionally, a multiplex fluorescent DNA labeling scheme for profiling the Oncosomes to detect aneuploidy is revealed. Finally, an ultra-fast in situ hybridization protocol with specialized buffers is made-known. By combining the efficient isolation of the Oncosomes from the plasma, with rapid hybridization of the fluorescent DNA probes with multiplex labeling scheme, it is possible to detect the presence of any human cancer from a liquid biopsy, using aneuploidy the hallmark of the human cancer.

### TECHNICAL FIELD

In some aspects, the present technology relates to a method and/or system for use in connection with isolating and profiling of extracellular vesicles (EVs) to detect human cancer. In some other aspects, the present technology relates to methods associated with isolating and profiling of exosomes for early detection and monitoring of all human cancers from peripheral blood. In yet other aspects, the present technology relates to methods associated with isolating and profiling of Oncosomes for early detection and monitoring of all human cancers from peripheral blood.

### BACKGROUND ART

Since a majority of the existing methods depend on genome sequencing, all of which have method failure rates ranging from 5-10%. Ultracentrifuges are very expensive instruments and only few research and commercial laboratories have the necessary infrastructure. Additionally, all current assays concentrate on the smallest exosomes which have only trace amounts of genomic DNA and therefore are unsuitable for profiling the hallmark trait i.e., the aneuploidy, of the cancer cells.

Some major drawback of currently known methods and/or systems are: 1) longer turnaround time (TAT); 2) higher cost; 3) limited resolution for early detection of cancer; 4) variability of sensitivity among different technologies to detect cancer; 5) high infrastructure cost; and 6) invasive procedures like tissue and bone marrow biopsy.

### DISCLOSURE OF TECHNOLOGY

In view of the foregoing disadvantages inherent in the known types of systems and/or methods at least some embodiments of the present technology provides a novel dismountable method and system for isolating and profiling of Oncosomes for early detection and monitoring of all human cancers from peripheral blood, and overcomes one or more of the mentioned disadvantages and drawbacks of the prior art. As such, the general purpose of at least some embodiments of the present technology, which will be described subsequently in greater detail, is to provide a new and novel method and system for isolating and profiling of Oncosomes for early detection and monitoring of all human cancers from peripheral blood which has all the advantages of the prior art mentioned herein and many novel features that result in a method and system for isolating and profiling of Oncosomes for early detection and monitoring of all human cancers from peripheral blood which is not anticipated, rendered obvious, suggested, or even implied by the prior art, either alone or in any combination thereof.

According to one aspect, the present technology can include a method for detecting and monitoring human cancer cells from peripheral blood. The method can include the steps of performing a centrifugation of a supernatant including plasma to pellet Oncosomes and separate all other exosomes into the supernatant. Performing in situ hybridization on the Oncosomes. Preparing a plurality of DNA probes such that each probe can be specific to a region of a human chromosome. Each of the DNA probes can have one to two different fluorescent tags attached to the respective the DNA probes. Each of the fluorescent tags can emit a separate color and such the DNA probes hybridize to specific regions of human chromosomes. Performing one or more simultaneous rapid in situ hybridizations of the DNA probes to the select regions of human chromosomes present in the Oncosomes to form hybridized Oncosomes. Determining a chromosomal ploidy status of the hybridized Oncosomes. Counting positively fluorescently stained the hybridized Oncosomes and determining the presence or absence of malignancy based on a number and the ploidy status of the hybridized Oncosomes.

According to another aspect, the present technology can include a method for isolation and profiling of Oncosomes for detecting and monitoring of human cancer cells from peripheral blood. The method can include the steps of performing a first centrifugation of plasma at a first speed to remove cell debris and apoptotic bodies, and to form a supernatant. Performing a second centrifugation of the supernatant at a second speed to pellet Oncosomes and separating all other exosomes into the supernatant. Resuspending the Oncosomes in phosphate buffered saline (PBS) and harvesting and fixing the Oncosomes to form fixed Oncosomes. Performing in situ hybridization on the fixed Oncosomes. Preparing a plurality of DNA probes such that each probe is specific to a region of a human chromosome. Each of the DNA probes can have one to two different fluorescent tags attached to the respective the DNA probes. Each of the fluorescent tags can emit a separate color and such the DNA probes hybridize to specific regions of human chromosomes. Performing one or more simultaneous rapid in situ hybridizations of the DNA probes to the select regions of human chromosomes present in the fixed Oncosomes to form fixed hybridized Oncosomes. Determining a chromosomal ploidy status of the fixed hybridized Oncosomes. Counting positively fluorescently stained the fixed hybridized Oncosomes and determining the presence or absence of malignancy based on a number and ploidy status of the Oncosomes.

According to another aspect, the present technology can include a method for isolation and profiling of Oncosomes for detecting and monitoring of human cancer cells from peripheral blood. The method can include the steps of separating the plasma from the peripheral blood by centrifugation using LymphoPrep density gradient solution. Performing a first centrifugation of plasma at a first speed to remove cell debris and apoptotic bodies, and to form a supernatant. Performing a second centrifugation of the supernatant at a second speed to pellet Oncosomes and separate all other exosomes into the supernatant. Resuspending the Oncosomes in phosphate buffered saline (PBS) and harvesting and fixing the Oncosomes by the use of KCl hypotonic solution and 3Metanol:1Acetic acid fixative respectively to form fixed Oncosomes. Dropping the fixed Oncosomes onto glass microscope slides for in situ hybridization on the fixed Oncosomes. Preparing a plurality of DNA probes such that each probe is specific to a region of a human chromosome. Each of the DNA probes can have one to two different fluorescent tags attached to the respective the DNA probes. Each of the fluorescent tags can emit a separate color and such the DNA probes hybridize to specific regions of human chromosomes. Performing one or more simultaneous rapid in situ hybridizations of the DNA probes to the select regions of human chromosomes present in the fixed Oncosomes to form fixed hybridized Oncosomes. Determining a chromosomal ploidy status of the fixed hybridized Oncosomes. Counting positively fluorescently stained the fixed hybridized Oncosomes and determining the presence or absence of malignancy based on a number and ploidy status of the Oncosomes.

In some embodiments, the plasma can be subjected to a first centrifugation at a first speed to remove cell debris and apoptotic bodies, and to form the supernatant.

In some embodiments, the supernatant can be subjected to a second centrifugation at a second speed to pellet the Oncosomes and separate all other exosomes into the supernatant.

In some embodiments, the second speed is greater than the first speed.

In some embodiments, prior to the in situ hybridization of the Oncosomes, the Oncosomes can be resuspended in phosphate buffered saline (PBS), and harvested and fixed to form fixed Oncosomes.

In some embodiments, the plasma can be separated from the peripheral blood by centrifugation using LymphoPrep density gradient solution.

In some embodiments, the harvesting and fixing of the Oncosomes can be performed by using a KCl hypotonic solution and 3Metanol:1Acetic acid fixative respectively.

In some embodiments, the in situ hybridization can include dropping the fixed Oncosomes onto glass slides.

In some embodiments, the glass slides can each have two separate chambers, with each of the chambers receiving the fixed Oncosomes.

In some embodiments, the step of counting positively fluorescently stained the fixed hybridized Oncosomes can be carried out for each of the two chambers on the glass slide.

In some embodiments, the ploidy status can be determined in the first chamber for chromosomes 5, 7, 8, 13, 15, 16, 17, 18, 21, 22, and sex chromosomes (X and Y) is determined, and the ploidy status is determined in the second chamber for chromosomes 1, 2, 3, 4, 6, 9, 10, 11, 12, 14, 19 and 20.

In some embodiments, the first speed of the first centrifugation can be 2,800 g.

In some embodiments, the second speed of the second centrifugation can be 9,000 g.

There has thus been outlined, rather broadly, features of the present technology in order that the detailed description thereof that follows may be better understood and in order that the present contribution to the art may be better appreciated.

Numerous objects, features and advantages of the present technology will be readily apparent to those of ordinary skill in the art upon a reading of the following detailed description of the present technology, but nonetheless illustrative, embodiments of the present technology when taken in conjunction with the accompanying drawings.

As such, those skilled in the art will appreciate that the conception, upon which this disclosure is based, may readily be utilized as a basis for the designing of other structures, methods and systems for carrying out the several purposes of the present technology.

It is therefore an object of the present technology to provide a new and novel method and system for isolating and profiling of Oncosomes for early detection and monitoring of all human cancers from peripheral blood that has all of the advantages of the known systems and/or methods and none of the disadvantages.

It is another object of the present technology to provide a new and novel method and system for isolating and profiling of Oncosomes for early detection and monitoring of all human cancers from peripheral blood that may be easily and efficiently manufactured and marketed.

An even further object of the present technology is to provide a new and novel method and system for isolating and profiling of Oncosomes for early detection and monitoring of all human cancers from peripheral blood that has a low cost of manufacture with regard to both materials and labor, and which accordingly is then susceptible of low prices of sale to the consuming public, thereby making such method and system for isolating and profiling of Oncosomes for early detection and monitoring of all human cancers from peripheral blood economically available to the buying public.

Still another object of the present technology is to provide a new method and system for isolating and profiling of Oncosomes for early detection and monitoring of all human cancers from peripheral blood that provides in the apparatuses and methods of the prior art some of the advantages thereof, while simultaneously overcoming some of the disadvantages normally associated therewith.

For a better understanding of the present technology, its operating advantages and the specific objects attained by its uses, reference should be made to the accompanying drawings and descriptive matter in which there are illustrated embodiments of the present technology. Whilst multiple objects of the present technology have been identified herein, it will be understood that the following description is not limited to meeting most or all of the objects identified and that some embodiments of the present technology may meet only one such object or none at all.

### BRIEF DESCRIPTION OF THE DRAWINGS

The technology will be better understood and objects other than those set forth above will become apparent when consideration is given to the following detailed description thereof. Such description makes reference to the annexed drawings wherein:

FIG. 1 is a representation showing the blood components before and after centrifugation with lymphoprep and Oncosomes floating into the plasma.

FIG. 2a is a microscope image of a tumor cell.

FIG. 2b is a microscope image of oncosome from the tumor cell in FIG. 2a, both showing similar genome profile after in situ hybridization with DNA probes.

FIG. 2c is a microscope image of a tumor cell.

FIGS. 2d and 2e are microscope images of Oncosomes produced from the tumor cell in FIG. 2c from K562 cell line, all at 60× magnification, with emphasis on the size difference between parental tumor cell and the Oncosomes.

FIG. 2f is a microscope image of a tumor cell from a solid cancer patient.

FIG. 2g is a microscope image of oncosome from the same patient in FIG. 2f both at 20× magnification, with emphasis on the size difference between the tumor cell and the oncosome.

FIG. 2h is a microscope image of a tumor cell from SKBR3 of a breast cancer cell line, at magnification shown as a reference.

FIG. 3 is a diagram showing how and where to analyze the cells with the highest hybridization efficiency along with the periphery of where the coverslip sat.

The same reference numerals refer to the same parts throughout the various figures.

### DETAILED DESCRIPTION OF THE TECHNOLOGY

Apart from the cumbersomeness, expensive costs and inaccurate results of known methods and systems, the present technology overcomes their disadvantages in a simple and cost-effective method.

While the above-described devices fulfill their respective, particular objectives and requirements, the aforementioned devices or systems do not describe a method and system for isolating and profiling of Oncosomes that allows for an early detection and monitoring of all human cancers from peripheral blood. An embodiment of the present technology deals with isolation and characterization of one class of extracellular vesicles (EVs). The present technology is an improvement on what currently exists, and is different from the existing sequence-based methods because it employs rapid in situ hybridization platform and DNA fluorescent probes for ploidy detection. The present technology further differentiates from existing gold standard of ultracentrifugation for the separation of extracellular vesicles produced by the cancer cells, by employing an inexpensive density gradient and sequential low speed centrifugations.

Also, the present technology can produce faster methods for detection and characterization of Oncosomes for the early detection of all human cancers from liquid biopsy, and/or a more practical and economical approach that can be employed in almost all laboratories unlike the existing methodology practiced by only few players.

Some advantages of the present technology are that it is better than the existing methods because of: 1) fast TAT; 2) lower cost; 3) ease of use in almost all laboratory setups without significant infrastructure expenditure; 4) comprehensive screening for aneuploidy of all chromosomes; and 5) detection of all human cancers at an early stage.

A need exists for a new and novel method and system for isolating and profiling of Oncosomes that can be used for early detection and monitoring of all human cancers from peripheral blood. In this regard, the present technology substantially fulfills this need. In this respect, the method and system for isolating and profiling of Oncosomes for early detection and monitoring of all human cancers from peripheral blood according to the present technology substantially departs from the conventional concepts and designs of the prior art, and in doing so provides an apparatus primarily developed for the purpose of early detection and monitoring of all human cancers from peripheral blood.

In the following description, for purposes of explanation and not limitation, specific details are set forth, such as particular embodiments, procedures, techniques, etc. in order to provide a thorough understanding of the present technology. However, it will be apparent to one skilled in the art that the present technology may be practiced in other embodiments that depart from these specific details.

It is known that cells communicate directly through physiological contact. However, other processes of communication exist, such as through the influence of soluble mediators such as growth factors, cytokines and chemokines. Still another process of communication exists, that being intercellular communication that permits the exchange of information between cells through extracellular vesicles (EVs). EVs are microscopic (50 nm-10 μM) phospholipid bilayer enclosed entities produced by virtually all eukaryotic cells. It is also known that cancer cells have the potential to utilize EVs in a specific manner. For example, cancer cell EVs overproduction presents benefits to tumor growth and metastasis, compared with neighboring healthy cells.

EVs can be defined based on their physical nature, size and biogenesis origin. Nevertheless, EVs can be classified as either endosomes or ectosomes. The endosome as an organelle comprises internal membranes within the mammalian cell that ultimately fuses with the cells' plasma membrane, forming multi-vesicular bodies (MVB). These are categorized as intraluminal vesicles (ILVs) when present in the cytoplasm, or as exosomes when released into the extracellular milieu. Endosomal vesicles typically range between 40 and 100 nm in diameter, whereas ectosomes are considerably larger, ranging from 100 nm to 10 μm. Ectosomes have also been referred to as microvesicles (MVs), microparticles (MPs), Oncosomes, shedding vesicles, exosome-like vesicles or nanoparticles.

A large scope and span of different terminology to describe EVs is currently present, including terms such as, but not limited to Oncosomes, exosomes, ectosomes, microvesicles, microparticles, shed vesicles, prostasomes, promininosomes, tolerosomes, apoptotic bodies, nanovesicles and several others.

The term “Oncosomes” can be utilized in the present technology to describe different aspects of EV release by cancer cells. EV terminology is sometimes reflective of EV cargoes. Oncosomes are 100-400 nm vesicles carrying abnormal and transforming macromolecules such as oncogenic proteins. In other cases, EVs are known as large Oncosomes (LO) since they are distinct from other EVs and typically 1-10 μm in size (21). Further, Oncosomes can be a membrane-derived microvesicle that is secreted by cancer cells and transfers oncogenic messages and protein complexes across cell borders.

With the above in mind, EVs are membrane-enclosed particles that contribute to tumor progression by establishing a tumor-supportive environment. Exosomes are nano-sized EVs that may have been implicated in angiogenesis, tolerogenic immune response, fibroblast activation, and preparation of the metastatic niche.

In some highly migratory cancer cells, there may be an exhibition in formation of nonapoptotic membrane blebs. It is known that “blebs” can be described as a bulge of the plasma membrane of a cell, characterized by a spherical or blister-like, bulky morphology. Membrane blebs are formed when plasma membrane is detached from underlying actin cytoskeleton.

Pinching off of these blebs results in the release of EVs, which may be referred to as “Oncosomes”.

An embodiment of the present technology solves the problem of the need to do:
1) massive parallel sequencing of the genome to detect cancer specific abnormalities;
2) to apply ultracentrifugation technologies to obtain extracellular vesicles produced by cancer cells; and
3) sophisticated and expensive imaging technologies such as computerized tomography (CT), magnetic resonance imaging (MRI) and positron emission tomography (PET) scans to identify cancer nodules.

In an embodiment, the present technology can utilize a simple density gradient such as Lymphoprep for the initial separation of the Oncosomes into the plasma. Next, the present technology can utilize a sequential centrifugation, first at a low speed of 2,800 g to pellet the cell debris and apoptotic bodies followed by a second centrifugation at a slightly higher centrifugation at 9,000 g to pellet the Oncosomes (the largest exosomes) leaving all other exosomes of various sizes in the supernatant. The present technology can utilize DNA fluorescent probes for landmarks on all 24 human chromosomes and rapid in situ hybridization to determine the ploidy of the Oncosomes. Since Oncosomes are ONLY produced by cancer cells and they mimic parental genome the present technology can utilize the “hallmark” of all cancer cells i.e., aneuploidy in detecting the presence of all human cancer(s) via the peripheral blood, the so-called liquid biopsy.

The term “aneuploidy” can be described as the occurrence of one or more extra or missing chromosomes leading to an unbalanced chromosome complement, or any chromosome number that is not an exact multiple of the haploid number, which is 23.

These basic hallmark capabilities, distinct and supplementary, are, but not limited to: (1) sustaining proliferative signaling; (2) evading growth suppressors; (3) enabling replicative immortality; (4) activating invasion and metastasis; (5) inducing angiogenesis; and (6) resisting cell death.

In an embodiment, the present technology can be an improvement on what currently exists. Accordingly, the present technology differs from what currently exists. In one aspect, the present technology is different from the existing sequence-based methods because it employs rapid in situ hybridization platform and DNA fluorescent probes for ploidy detection. The present technology is different from the gold standard of ultracentrifugation to isolate the EVs produced by the cancer cells, by employing an inexpensive density gradient and sequential low speed centrifugations. It is better than the existing methods because of: 1) fast TAT; 2) lower cost; 3) ease of use in almost all laboratory setups without significant infrastructure expenditure; 4) comprehensive screening for aneuploidy of all chromosomes; and 5) detection of all human cancers at an early stage.

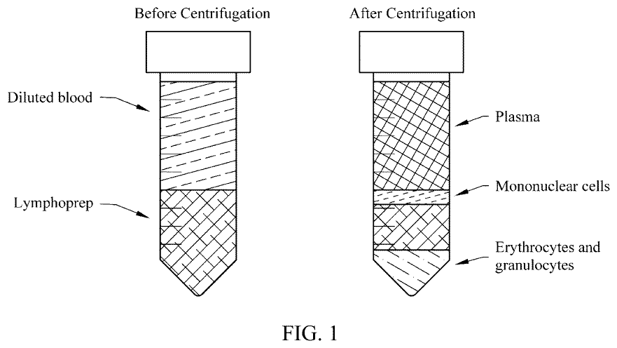

In an embodiment, the present technology can include the following different steps.
1. Obtain peripheral blood and separate plasma using the density gradient LymphoPrep, if needed use stabilizers for transport.
2. Separate cell debris and apoptotic bodies by low-speed centrifugation of plasma (see FIG. 1). Save supernatant.
3. Isolate Oncosomes by pelleting by centrifugation. Discard supernatant.
4. Suspend the Oncosome pellet in phosphate buffered saline (PBS) and harvest the cell button by hypotonic (KCl) and fixative treatments freshly prepared Methanol: Acetic acid 3:1 ratio.
5. Drop the resuspended pellet solution onto two chambers on a glass slide.
6. Prepare or obtain Fluorescent labelled DNA probes for specific genomic targets on individual chromosomes such as telomeres, centromeres, and specific loci.
7. Perform rapid in situ hybridization using the fluorescent DNA probe mixes on each chamber respectively, for ploidy detection.
8. Enumerate the number of Oncosomes in each chamber.
9. Determine the ploidy status of the Oncosomes in each chamber.
10. Complete the analysis:
   a) Presence or absence of Oncosomes;
   b) Enumeration (quantitation) of Oncosomes; and
   c) Ploidy status Normal or abnormal, of Oncosomes

In an embodiment, step 2 can be operated at, but not limited to, a low-speed centrifugation of 2800 g.

In an embodiment, step 3 can be operated at, but not limited to, a centrifugation speed of 9,000 g.

In an embodiment, the Fluorescent labelled DNA probes in step 6 can be obtained from or provided by InteGen LLC.

An embodiment of the present technology describes the relationship between the various components as follows:

Step 1 is a pre-requisite for step 2. In step 1, the plasma can be separated from the whole blood and in then in step 2, the cell debris is removed. Oncosomes are isolated in step 3. Step 4 is an integral part of step three, where the Oncosomes are prepared for further characterization. Step 4 is a pre-requisite for step 5 where the cell suspension is deposited onto the glass slide. In Step 6, Fluorescent labelled DNA probes are made or obtained from InteGen LLC for specific genomic targets on individual chromosomes such as telomeres, centromeres, and specific loci. This is a pre-requisite for rapid Fluorescence in situ hybridization (FISH), which is carried out in the next step 7. Enumeration of Oncosomes is carried out in step 8 for each chamber on the glass slide. In step 9, the ploidy status is determined from the DNA probe hybridization results. In the first chamber the ploidy status for chromosomes 5, 7, 8, 13, 15, 16, 17, 18, 21, 22, and sex chromosomes (X and Y) is determined and in the second chamber the ploidy status for chromosomes 1, 2, 3, 4, 6, 9, 10, 11, 12, 14, 19 and 20 is established. Finally in the last step (step 10), the analysis is completed by documenting the enumeration status as well as the ploidy status of the Oncosomes. Based on the results of this analysis, a determination of whether a tumor exists in the body is made.

In another embodiment, the process of how the present technology works is described.

There are basically four steps associated with the present technology: 1) Efficient separation of plasma from the suitably collected blood sample; 2) Better isolation and enrichment of Oncosomes from the plasma; 3) Multiplex fluorescent labelling of DNA probes; and 4) rapid in situ hybridization.

There are many ways to obtain plasma from a suitably collected blood sample. The simple and easy way is simply let the blood sample stand still upright for few hours and the top layer will be the plasma. However, for a rapid turnaround addition of a density gradient followed by centrifugation would accelerate the process of obtaining plasma. There are many density gradients commercially available for separation of blood components. In the present technology and in the exemplary, LymphoPrep was chosen to efficiently isolate the plasma.

The field of Exosomes is a decade old and numerous technologies have been developed during this period to isolate exosomes, which are the smallest of the extracellular vesicles produced by various cells in the body. A partial list of these technologies includes Ultracentrifugation, Size Exclusion Chromatography (SEC), Ultra filtration, Various precipitation methods, immuno magnetic separation etc.

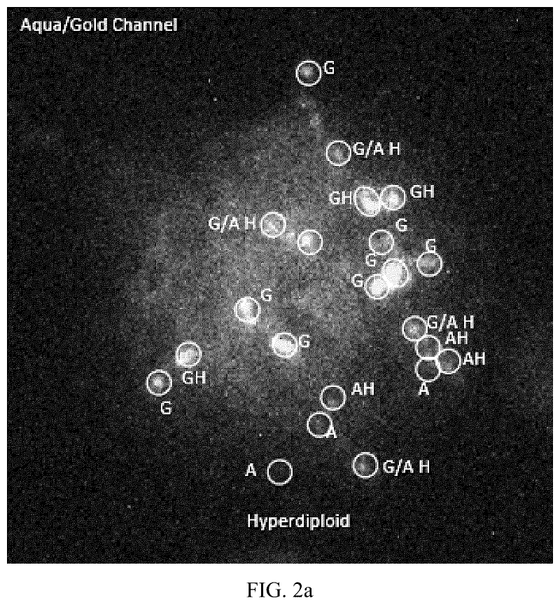

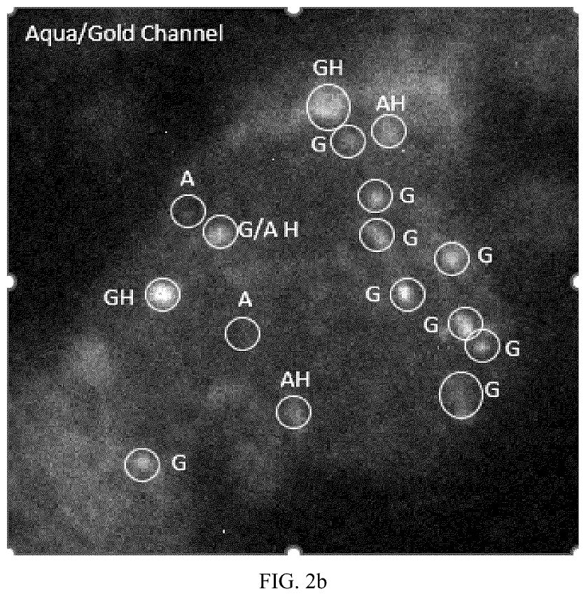

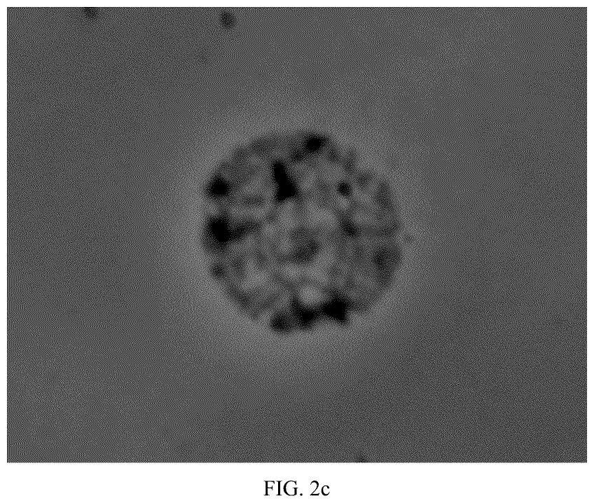

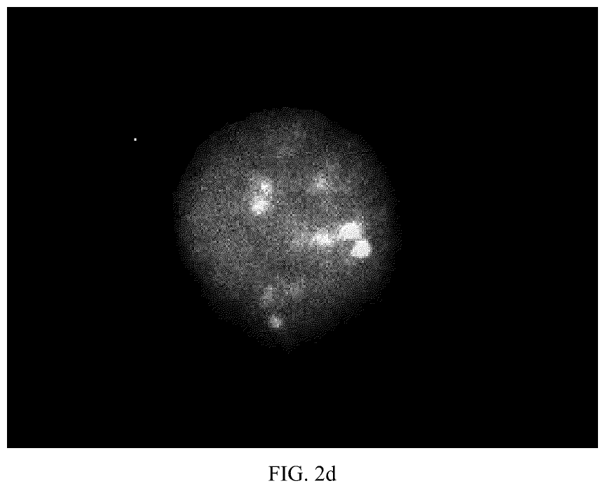

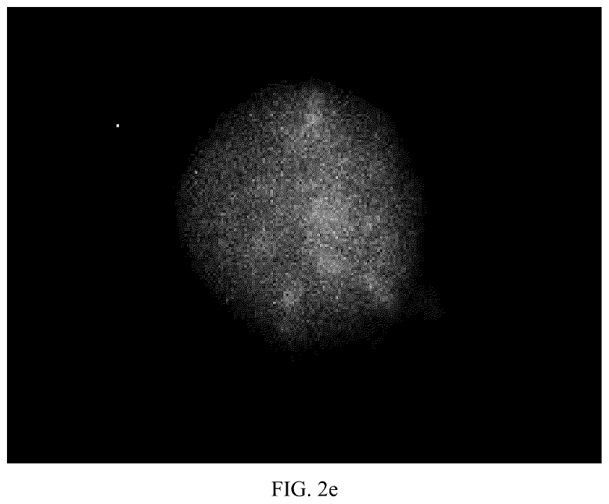

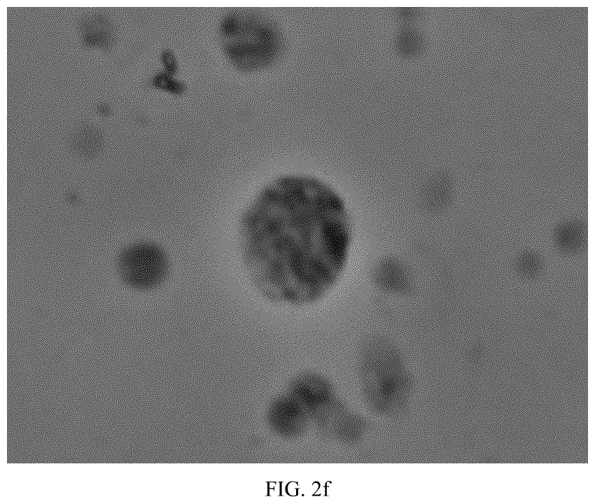

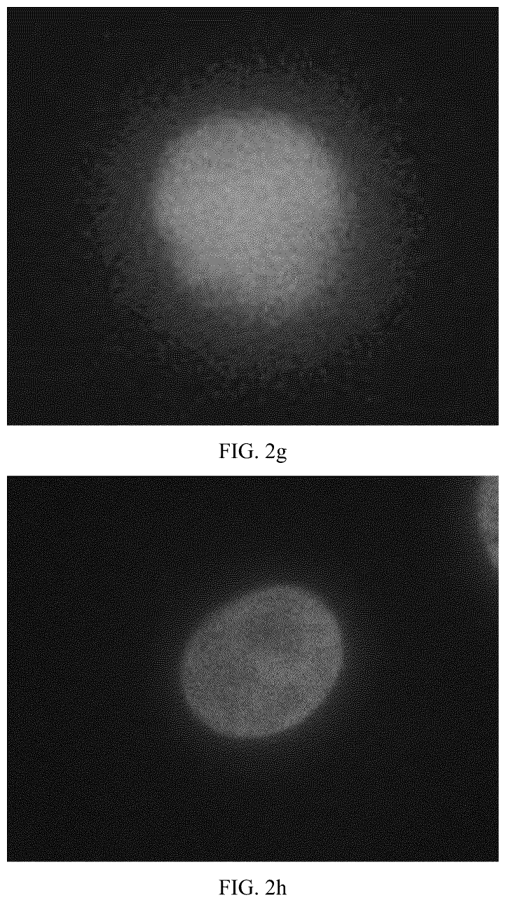

The cargo of exosomes includes various proteins, mRNA, miRNA and few other macro molecules. Numerous researchers have used different components of this exosome cargo in their efforts to develop assays for detecting solid tumors from liquid biopsy. Very little attention has been focused on the largest of the exosomes, namely Oncosomes which are ONLY produced by the cancer cells and contain whole genomic DNA, whereas exosomes contain very little, if any, of genomic DNA. Since the present technology relies on the detection of ploidy, which is based on the whole genomic DNA, Oncosomes are the only suitable extracellular vesicles to determine the aneuploid status which is the hallmark of all human solid tumors. Therefore, if one can successfully isolate and characterize the Oncosomes, one can detect the cancer from liquid biopsy. The simple sequential low to medium speed centrifugation steps utilized in the present technology guarantee the maximum recovery of Oncosomes. The tumor cells recovered from the buffy coat after the initial density gradient centrifugation serve as reference material to compare the physical and genetic characteristics between the Oncosomes and the tumor cells (see FIGS. 2a, 2c, 2f) confirming the validity of the use of Oncosomes as markers for detection of any cancer from the peripheral blood.

The common practice in the diagnostic workup of in situ hybridization is to use one or just a few, generally less than four, fluorescent DNA probes to investigate specific targets in the genome. This approach falls way short of obtaining the entire ploidy information in a single assay. The multiplex approach utilized in the present technology guarantees that each Oncosome can be queried for its ploidy status in a single assay with 12 chromosomes assayed in each chamber on the slide and by combining the results of the two chambers, one can get the ploidy status of the whole sample in a single assay without the need for any special equipment and software than a simple fluorescent microscope equipped with few filter cubes to detect all colors emitted by the multiplex DNA probes.

Turnaround time (TAT) plays a critical role in any diagnostic workup. Historically in situ hybridization protocols utilized an overnight time period to get the results. In an embodiment, the present technology can include a rapid hybridization protocol is utilized with results in as little as 15 minutes to less than an hour. Combining the efficient separation of plasma, maximum recovery of Oncosomes, multiplex DNA probe labelling and rapid in situ hybridization technology one can successfully determine the presence or absence of a cancer from a noninvasive sample collection such as a peripheral blood in less than three hours.

In yet another embodiment, the way the present technology can be made is described.

Several vacutainer tubes are commercially available for drawing the peripheral blood. For the purpose of the present technology and in the exemplary, only tubes with a proper anti-coagulant so that plasma can be separated will be used. There are many suitable blood collection tubes for this purpose such as EDTA purple or lavender tubes. Tubes designed for serum collection should be avoided. These generally have red or black tops. There are many density gradient solutions available. But the Lymphoprep with, but not limited to, a density of 1.077 g/ml is suited for efficiently separating the plasma as well as buffy coat which retains all mononuclear cells including the tumor cells, at a centrifugation of 1,200 g for 10 minutes at room temperature. Since the Oncosomes are smaller than any of the mono nuclear cells including the tumor cells, they float to the top layer (plasma) after the density gradient centrifugation. In order to get rid of cell debris an initial centrifugation at 2,800 g for 10 min, followed by a slightly higher centrifugation of the supernatant at 9,000 g for 15 min will pellet the Oncosomes leaving all other exosomes in the supernatant. One can get the Oncosomes by resuspending the pellet in PBS. To process them further for characterization, the traditional cytogenetic method of fixation using freshly prepared methanol: acetic acid with a 3:1 ratio is employed. Once the Oncosomes are fixed, the suspension is dropped onto a glass slide in two separate areas/chambers for in situ hybridization. There are many commercial sources for obtaining BAC probes and for the present technology BACPAC library resource is utilized to get the needed BACs. To generate the BAC DNA, several commercial BAC extraction protocols are available including propagation of BAC cultures. Labeling of BAC probes can be done by many protocols available, but for the present technology Nick translation will be utilized with various fluorescent dNTPs.

Cytogenetics involves the examination of chromosomes to identify structural abnormalities. Chromosomes of a dividing human cell can be analyzed clearly in white blood cells, specifically T lymphocytes, which are easily collected from blood.

Fixation is a critical step in chromosome study. It is the process by which tissues and their components are fixed selectively at a particular stage thereby respective divisional stages are arrested. Fixation in chromosome study brings about blocking of cell divisions and enables the preservation of the structural integrity of nucleic acid and protein of the chromosomes. It can be appreciated that cell fixation aims to maintain cells or cellular components in life-like state, preventing unexpected changes by preserving essential chemical and physical characteristics of cells for further observation.

In order to study the genome ploidy of each Oncosome present in each of the chambers on the glass slide, a multiplex hybridization protocol will be utilized as detailed in Table 1. There are many in situ hybridization protocols in the public domain and most employ an overnight hybridization time. Some vendors claim rapid hybridization from one to several hours. The procedures employed in the exemplary in the present technology with specialized hybridization buffers allow a very rapid hybridization with results in as little as 15 minutes to less than an hour. Details of which are listed in the Rapid FISH protocol procedure and protocol.

TABLE 1

| | PL1 | PL2 |
|---|---|---|
| Green | 13q11, 16q11 | 3p11.1, 19q11 |
| Green Hybrid | Yq11.1 | 6q11.1 |
| Red | 21q22.13 | 9q34 |
| Red Hybrid | Xp11.1 | 11q11 |
| Red/Green Hybrid | 18q11.1 | 10q11 |
| Gold | 22q11, 15q11 | 1q12, 20q11 |
| Gold Hybrid | 5q11 | 4q11 |
| Aqua | 7q11 | 2q11.1 |
| Aqua Hybrid | 8q11 | 12p11.1 |
| Gold/Aqua Hybrid | 17q11 | 14q11 |

### Rapid FISH Protocol (15 Minutes)

### Solutions:

2×SSC, pH 7.0
For 500 mL: Add 50 mL 20×SSC, to 450 mL type 1 water. pH to 7.0.
Store up to six months at room temperature.

### 2×SSC/0.1% NP40, pH 7.0-7.5

For 1,000 mL: To a beaker add 100 mL SSC to 400 mL type 1 water. Heat the solution on high while mixing with stir bar for 20-30 minutes. Add 1 mL NP40 to beaker. Stir solution until NP40 is completely dissolved. Adjust pH to 7.0-7.5. Add type 1 water to bring final volume to 1,000 mL. Store up to six months at room temperature.
0.4×SSC/0.3% NP40, pH 7.0-7.5
For 1,000 mL: To a beaker mix thoroughly 20 mL 20×SSC (pH 5.3) to 950 mL type 1 water. Heat the solution on high while mixing with a stir bar for 20-30 minutes. Add 3 mL NP-40. Stir solution until NP-40 is completely dissolved. Adjust pH to 7.0-7.5. Add type 1 water to bring final volume to 1,000 mL. Store up to six months at room temperature.
20×SSC, (3M Sodium Chloride, 0.3M Sodium Citrate), pH 5.3
For 400 mL: add 70.125 g of Sodium Chloride (NaCl) and 35.292 g Sodium Citrate to 300 mL type 1 water. Adjust pH to 5.3 and bring to a final volume of 400 mL with type 1 water. Sterilize the solution by autoclaving. Store up to six months at room temperature.

### 12. Rapid Hybridization Procedure

The ThermoBrite will not be used according to your lab's standard protocols. Hybridization will either not work or not be optimal unless these steps are followed. Wet moisture strips in ddH20 prior to hybridization. Place them in the internal slots of the hybridization chamber as you normally would. NOTE: The proper amount of humidity/internal condensation is critical to successfully performing the 15 minute hybridization. The hybridization will fail if the amount of water added is too low. Please consult with InteGen if you are not using a standard ThermoBrite.

### 12.1 Setup & Hybridization:

Setup a Melt/Hyb program in the ThermoBrite.
Melt: 82° C. (2 Minutes)
Hyb: 45° C. (15 Minutes)

| | |
|---|---|
| 12.1.1 | Place the desired probes on a 37° C. heat block and allow them |
| | to warm. Mix by vortexing and place back on heat block until |
| | ready to use. |
| 12.1.2 | Add 0.5 uL per area of hybridization of each desired probe mix |
| | to slide where cells were dropped. |
| 12.1.3 | Place 5 mm circular coverslips on probe and allow probe to |
| | completely cover the area of coverslip. Do not press down or |
| | slide coverslip. Do not seal coverslips with rubber cement. |
| 12.1.4 | Pipet 300-400 uL ddH20 onto the hyb chamber surface. |
| 12.1.5 | Carefully place the slide on top of the water. You will see the |
| | water spread across the bottom of the slide. |
| 12.1.6 | NOTE: Do not press slides together in the hybridization |
| | chamber. Close cover and start desired program. |
| | Start program: Denature 82° C. (2 Minutes) |
| | Hybridize: 45° C. (15 Minutes) |

### 12.2 Post Hybridization Washes

| | |
|---|---|
| 12.2.1 | When the hybridization is complete, carefully remove the |
| | coverslip using forceps. If coverslips are difficult to remove, |
| | dip slide in 2XSSC briefly and then remove coverslips. |
| 12.2.2 | Wash slides with 0.4X SSC/0.3% NP40 at 69° C. thoroughly for |
| | 2 minutes. Agitate slides 3-5 seconds as you place them in and |
| | also when you remove them from the jar. |
| 12.2.3 | Wash slides again with gentle agitation (as before) in 2X |
| | SSC/0.1% NP40 at room temperature for 1 minute. |
| 12.2.4 | Allow excess solution to drain off from the slides by touching |
| | the ends of the slide onto Kim Wipe. |
| 12.2.5 | Let slides air dry or fan dry. Add sufficient Slow Fade Gold |
| | Antifade to center of slide to ensure area is covered when |
| | coverslip is placed (N 18 to 20 uL for a 22 × 50 coverslip and |
| | 8 uL for a 22 × 22 coverslip. You may draw circles in the areas |
| | of interest on the back of the slide before viewing. |
| 12.2.6 | Cover slide with a glass cover slip. Blot to get rid of air |
| | bubbles and excess antifade. |
| 12.2.7 | Store slides for viewing in dark area or store for long term in |
| | 4° C. with a slide mailer. |

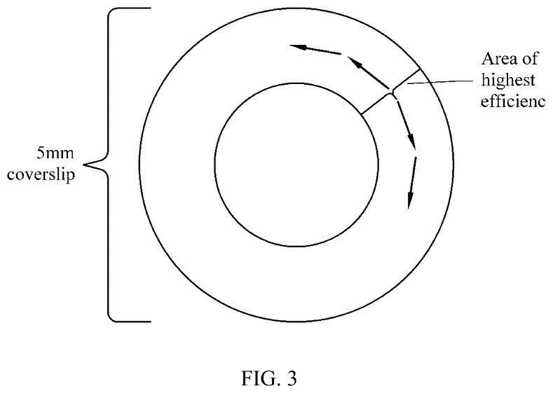

### 12.3 Area of Hybridization

| | |
|---|---|
| 12.3.1 | The 15 minute rapid hybridization yields the best results on the |
| | periphery of where the coverslip sat. FIG. 3 is a diagram to show |
| | how and where to analyze the cells with the highest |
| | hybridization efficiency. Analyze around the edge. |

In an embodiment of the present technology, the characteristics of the Oncosomes are elucidated.
1) Density gradient separates all mononuclear cells, white cells (lymphocytes and monocytes) and platelets, into the buffy coat and the plasma contains the oncosomes and proteins (see FIG. 1).
2) Centrifugation of plasma at low speed (2,800 g) will pellet any cell debris, apoptotic bodies and the supernatant contains proteins and exosomes of all sizes (oncosomes are the largest). 3) The tumor cells which are generally larger than white cells are trapped in the buffy

coat.
4) Further centrifugation of the supernatant at higher speed (9,000 g) will pellet large exosomes. The smaller exosomes will remain in the supernatant.
5) Genomic DNA is largely present in the larger exosomes (Oncosomes) and very little exists in the exosomes.
6) Exosomes are so small, they cannot be visualized under the ordinary light microscope and after hybridization with DNA probes, they are still not visible under fluorescent microscope due to very little DNA.
7) On the other hand, large exosomes (oncosomes) contain whole genomic DNA (all 24 chromosomes) and after DNA hybridization with 24 chromosome probes, are visible under the fluorescent microscope. However, they are still smaller than other nucleated cells i.e., WBC, tumor cells etc. (FIGS. 2a-2h).
8) No other nucleated cells are present in plasma. Apoptotic bodies may contain fragmented DNA, and these are usually produced by the dying cells, including cancer cells. They are typically larger and present in the buffy coat fraction or pellet during the first low speed centrifugation. But the main characteristic is they do not contain full chromosomes while oncosomes do.
9) While apoptotic bodies are generally produced by the dying cells, in the case of tumors they are produced because of chemo or radiation therapy, oncosomes are produced by “healthy” tumor cells.
10) Oncosomes mimic the parental DNA profile and do not contain intact organelle like Golgi apparatus etc.

### EXAMPLES

### Example 1

In an embodiment of the present technology, an example of the presence of sufficient number of oncosomes before the onset of the disease is described.

About 72 oncosomes were observed in the plasma from the original 3 cc of blood in a patient clinically considered normal, but with some chest pain. After 5½ weeks, pathological confirmation of invasive breast carcinoma was made from a tissue biopsy. This illustrates the higher sensitivity of the oncosome isolation and enumeration methods described in the present technology.

### Example 2

In an embodiment of the present technology, an example of the presence of sufficient number of oncosomes confirming the presence of the disease is described.

In a patient with elevated PSA and clinical confirmation of prostate cancer, from 3 cc of peripheral blood, around 45 oncosomes were present in the plasma confirming the utility of the methods described in the present technology for cancer diagnostics.

### Example 3

In an embodiment of the present technology, an example of the decline of the number of oncosomes during the treatment of the disease is described.

In a patient with squamous cell cancer before the treatment started from 3 cc of peripheral blood, a count of 600 oncosomes was obtained. A gradual decline of the number of oncosomes was observed as follows: At about 5 weeks the number of oncosomes from 3 cc of blood was about 500, at 8 weeks 25 and 12 weeks 4, respectively, illustrating the utility of the present technology described in this application in monitoring the efficacy of the treatment for cancer.

### Example 4

In an embodiment of the present technology, an example of the absence of the oncosomes confirming the remission status after the treatment of the disease is described.

A patient with colon cancer had 72 oncosomes in the plasma from the original 3 cc's of blood before treatment. After 12 weeks of treatment with partial colectomy and radiation, a count of zero oncosomes was observed confirming the clinical remission status. This again proves the utility of the present technology in monitoring various treatment modalities of cancer.

### Example 5

In an embodiment of the present technology, an example of the absence of the oncosomes indicating the absence of the disease is described.

As part of a screening program, an elderly person clinically considered normal had a count of zero oncosomes in the plasma from the original 3 cc of peripheral blood illustrating the sensitivity and utility of the present technology in screening for cancer in high risk population such as people older than 50 years of age.

### INDUSTRIAL APPLICABILITY

The present technology has major applications in the fields of clinical oncology and diagnostics.

In order to detect all human cancers at any stage using the liquid biopsy i.e., the peripheral blood, one with skill in the art can perform the following tasks:
1) Separate the plasma from the peripheral blood using LymphoPrep.
2) Remove cell debris by low speed centrifugation.
3) Isolate Oncosomes from the rest of the exosomes by medium speed centrifugation.
4) Fix the Oncosome preparation for in situ hybridization.
5) Prepare Fluorescent DNA BAC probes for specific targets on all human chromosomes by multiplex labeling protocols.
6) Perform rapid in situ hybridization with all 24 chromosome probe mixes.
7) Enumerate and analyze the ploidy status of the Oncosomes to determine the presence of a cancer as well as monitor the efficacy of various treatment modalities.

The examples provided above illustrate the industrial utility of the present technology.

While embodiments of the method and system for isolating and profiling of Oncosomes for early detection and monitoring of all human cancers from peripheral blood have been described in detail, it should be apparent that modifications and variations thereto are possible, all of which fall within the true spirit and scope of the present technology. With respect to the above description then, it is to be realized that the optimum dimensional relationships for the parts of the present technology, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present technology. And although early detection and monitoring of all human cancers from peripheral blood have been described, it should be appreciated that the method and system for isolating and profiling of Oncosomes for early detection and monitoring of all human cancers from peripheral blood herein described is also suitable for detection of other medical conditions.

Therefore, the foregoing is considered as illustrative only of the principles of the present technology. Further, since numerous modifications and changes will readily occur to those skilled in the art, it is not desired to limit the present technology to the exact construction and operation shown and described, and accordingly, all suitable modifications and equivalents may be resorted to, falling within the scope of the present technology.

### REFERENCES

1. Tatyana Vagnera, Cristiana Spinelli, Valentina R. Minciacchi, Leonora Balajf, Mandana Zandiana, Andrew Conleyb, Andries Zijlstrag, Michael R. Freeman, Francesca Demichelisi, Subhajyoti Dej, Edwin M. Posadask, Hisashi Tanakaa, and Dolores Di Vizio.Large extracellular vesicles carry most of the tumour DNA circulating in prostate cancer patient plasma J Extracell Vesicles. 7 (1): 1505403, 2018.
2. Babu R, Van Dyke D L, Papa S, Fuentes E, Fuentes S, Kopuri S, Williamson C, Liu M, Dev V G, Tepperberg J, Schwartz S, Papenhausen P and Koduru P. Development and Validation of 15 minute FISH hybridization technology for interphase and metaphase cytogenetic samples. Platform presentation at American Cytogenetics Conference, June 2018.
3. Ramesh Babu 15-minute InstaFISH. Workshop and Live Demonstration. Cancer Genetics, Consortium Annual Meeting, August 2018.
4. D. Di Vizio, et al., Large oncosomes in human prostate cancer tissues and in the circulation of mice with metastatic disease, Am. J. Pathol. 181, 1573-1584, 2012.
5. Nobuyoshi Kosaka, Akiko Kogure, Tomofumi Yamamoto, Fumihiko Urabe, Wataru Usuba, Marta Prieto-Vila, and Takahiro Ochiya. Exploiting the message from cancer: the diagnostic value of extracellular vesicles for clinical applications. Experimental & Molecular Medicine 51:31, 2019.
6. Kristin A. Knouse, Teresa Davoli, Stephen J. Elledge, and Angelika Amon. Aneuploidy in Cancer: Seq-ing Answers to Old Questions Annu. Rev. Cancer Biol. 1:335-54, 2017.
7. LymphoprepTM Density gradient medium for the isolation of mononuclear cells. STEMCELL Technologies Inc.
8. BACPAC library https://bacpacresources.org/.
9 M. Hristov, W. Erl, S. Linder and P. C. Weber, Apoptotic bodies from endothelial cells enhance the number and initiate the differentiation of human endothelial progenitor cells in vitro, Blood, 104, 2761-2766, 2004.

1: A method for detecting and monitoring of human cancer cells from peripheral blood, the method comprising the steps of:
a) performing a centrifugation of a supernatant including plasma to pellet Oncosomes and separate all other exosomes into the supernatant;
b) performing in situ hybridization on the Oncosomes;
c) preparing a plurality of DNA probes such that each probe is specific to a region of a human chromosome, each of the DNA probes having one to two different fluorescent tags attached to the respective the DNA probes, each of the fluorescent tags emits a separate color and such the DNA probes hybridize to specific regions of each of the twenty-four human chromosomes;
d) performing one or more simultaneous rapid in situ hybridizations of the DNA probes to the select regions of the twenty-four human chromosomes present in the Oncosomes to form hybridized Oncosomes;
e) determining a chromosomal ploidy status of the hybridized Oncosomes; and
f) counting positively fluorescently stained the hybridized Oncosomes and determining the presence or absence of malignancy based on a number and the ploidy status of the hybridized Oncosomes.

2: The method of claim 1, wherein the plasma is subjected to a first centrifugation at a first speed to remove cell debris and apoptotic bodies, and to form the supernatant.

3: The method of claim 2, wherein the supernatant is subjected to a second centrifugation at a second speed to pellet the Oncosomes and separate all other exosomes into the supernatant, and wherein the second speed is greater than the first speed.

4: The method of claim 3, wherein, prior to the in situ hybridization of the Oncosomes, the Oncosomes are resuspended in phosphate buffered saline (PBS), and harvested and fixed to form fixed Oncosomes.

5: The method of claim 4, wherein the plasma is separated from the peripheral blood by centrifugation using LymphoPrep density gradient solution, and wherein the harvesting and fixing of the Oncosomes is performed by using a KCl hypotonic solution and 3Metanol:1 Acetic acid fixative respectively.

6: A method for isolation and profiling of Oncosomes for detecting and monitoring of human cancer cells from peripheral blood, the method comprising the steps of:
a) performing a first centrifugation of plasma at a first speed to remove cell debris and apoptotic bodies, and to form a supernatant;
b) performing a second centrifugation of the supernatant at a second speed to pellet Oncosomes and separate all other exosomes into the supernatant;
c) resuspending the said Oncosomes in phosphate buffered saline (PBS) and harvesting and fixing the Oncosomes to form fixed Oncosomes;
d) performing in situ hybridization on the said fixed Oncosomes;
e) preparing a plurality of DNA probes such that each probe is specific to a region of a human chromosome, each of the DNA probes having one to two different fluorescent tags attached to the respective said DNA probes, each of the fluorescent tags emits a separate color and such said DNA probes hybridize to specific regions of each of the twenty-four human chromosomes;
f) performing one or more simultaneous rapid in situ hybridizations of said DNA probes to said select regions of the twenty-four human chromosomes present in the said fixed Oncosomes to form fixed hybridized Oncosomes;
g) determining a chromosomal ploidy status of said fixed hybridized Oncosomes; and
h) counting positively fluorescently stained said fixed hybridized Oncosomes and determining the presence or absence of malignancy based on a number and the ploidy status of said fixed hybridized Oncosomes.

7: The method of claim 6, wherein the plasma is separated from the peripheral blood by centrifugation using LymphoPrep density gradient solution.

8: The method of claim 6, wherein the harvesting and fixing of the Oncosomes is performed by using a KCl hypotonic solution and 3Metanol:1Acetic acid fixative respectively.

9: The method of claim 6, wherein the in situ hybridization includes dropping the said fixed Oncosomes onto glass slides.

10: The method of claim 9, wherein the glass slides has two separate chambers, with each of the chambers receiving the fixed Oncosomes.

11: The method of claim 10, wherein step h) is carried out for each of the two chambers on the glass slide.

12: The method of claim 11, wherein the ploidy status is determined in the first chamber for chromosomes 5, 7, 8, 13, 15, 16, 17, 18, 21, 22, and sex chromosomes (X and Y) is determined, and the ploidy status is determined in the second chamber for chromosomes 1, 2, 3, 4, 6, 9, 10, 11, 12, 14, 19 and 20.

13: The method of claim 6, wherein the first speed of the first centrifugation is 2,800 g.

14: The method of claim 6, wherein the second speed of the second centrifugation is 9,000 g.

15: A method for isolation and profiling of Oncosomes for detecting and monitoring of human cancer cells from peripheral blood, the method comprising the steps of:
a) separating the plasma from the peripheral blood by centrifugation using LymphoPrep density gradient solution;
b) performing a first centrifugation of plasma at a first speed to remove cell debris and apoptotic bodies, and to form a supernatant;
c) performing a second centrifugation of the supernatant at a second speed to pellet Oncosomes and separate all other exosomes into the supernatant;
d) resuspending the said Oncosomes in phosphate buffered saline (PBS) and harvesting and fixing the Oncosomes by the use of KCl hypotonic solution and 3Metanol:1Acetic acid fixative respectively to form fixed Oncosomes;
e) dropping the said fixed Oncosomes onto glass microscope slides for in situ hybridization on the said fixed Oncosomes;
f) preparing a plurality of DNA probes such that each probe is specific to a region of a human chromosome, each of the DNA probes having one to two different fluorescent tags attached to the respective said DNA probes, each of the fluorescent tags emits a separate color and such said DNA probes hybridize to specific regions of each of the twenty-four human chromosomes;
g) performing one or more simultaneous rapid in situ hybridizations of said DNA probes to said select regions of the twenty-four human chromosomes present in the said fixed Oncosomes to form fixed hybridized Oncosomes;
h) determining a chromosomal ploidy status of said fixed hybridized Oncosomes; and
i) counting positively fluorescently stained said fixed hybridized Oncosomes and determining the presence or absence of malignancy based on a number and the ploidy status of said fixed hybridized Oncosomes.
